(19) United States
(12) Patent Application Publication
(10) Pub. No.: US 2025/0002831 A1
(43) Pub. Date: Jan. 2, 2025

(54) TOOL FOR SAND REMOVAL FROM BIOGAS REACTORS AND OTHER EQUIPMENT

(71) Applicant: Sandcatch Solutions AS, Bryne (NO)

(72) Inventor: Trygve HUSVEG, Varhaug (NO)

(73) Assignee: Sandcatch Solutions AS, Bryne (NO)

(21) Appl. No.: 18/710,872

(22) PCT Filed: Nov. 15, 2022
(86) PCT No.: PCT/NO2022/050262

§ 371 Date: May 16, 2024

(30) Foreign Application Priority Data
Nov. 18, 2021 (NO) 20211383

(51) Int. Cl. C12M 1/00

(52) U.S. Cl. CPC C12M 29/06; C12M 23/50

(57) ABSTRACT

Tool (1) for sand removal from biogas reactors (2) and other containments, feasible for sand removal while the biogas reactor or other containment is in operation at elevated pressure and temperature. The tool is distinguished in that it comprises: a number of nozzles (3) fluidly coupled to a high pressure liquid source for high pressure liquid injection, in operation directed at volumes (4) with solid, semi solid or soft sand, wherein high-pressure liquid injection through the nozzles creates a flowable sand slurry, a flowable slurry outlet opening (6) facing the flowable slurry created by the nozzles, and two flow paths, integrated in the tool, each flow path being fluidly coupled to one or both of the nozzles and the outlet opening, wherein a first flow path is for high pressure liquid to be injected through the nozzles and/or to create suction effect in the outlet opening, and wherein a second flow path is for removal of slurry collected through the outlet opening.

### TECHNICAL FIELD

The present invention relates to equipment and methods for removal of sand or other materials from biogas reactors and other equipment. More specifically, the invention relates to removal of settled sand that may have become more or less solidified, or other unwanted material that may accumulate in equipment during operation, while said equipment can be in operation at elevated pressure and temperature.

### BACKGROUND ART

Sand settles in numerous types of equipment. The sand must typically be removed for not reducing the capacity of the equipment and not reducing the reliability and service life of equipment further downstream in a process.

A very relevant example of equipment is biogas reactors, used to produce biogas from organic waste or manure. The organic waste or manure may contain sand and other material not active in the biogas production process.

A biogas reactor is typical of volume 1000 m³ to 6000 m³. In practice, after some period of operation, a layer of more or less solidified sand, in average 1 m to 3 m thick, typically fill the bottom part of the reactor. This means that about 10 to 20% or often more of the reactor volume is filled with sand. The capacity of the reactor is reduced accordingly. In addition, the reactor must be shut down when sand shall be removed. Typically, such shut down results in about one to two months out of operation until full biogas reactor operation is resumed. Removal of sand and resulting shut down is required at an interval typically of about every second or third year. The sand sedimented out in the tank over time may transform from soft sand to semi solid sand to a more solid-like material. Longer periods between shut down for sand removal may therefore increase the problem.

Apparently, no equipment exists allowing the sedimented sand, more or less like a solid or semi solid material, to be taken out from a biogas reactor without stopping operation of the reactor. A reason may be the technical and practical challenges faced. Some biogas reactors are operated at elevated pressure and temperature, from a few mbar and at least up to pressure of 25 bar and temperature up to 150°. Elevated temperatures, pressures and abrupt changes thereof are essential operation steps of some biogas or other production processes.

Searching has revealed only one patent publication of some relevance for the underlying problem of the present invention, namely U.S. Pat. No. 7,240,681 B2, relating to a mobile apparatus for removing formation sand from an oil storage tank. According to U.S. Pat. No. 7,240,681 B2, two separate pipes are flanged to the tank wall. A first pipe is inside the tank, with one end coupled to a slurry formation tool and an opposite end coupled to a first flange in the tank wall. A second pipe is outside the tank and is coupled to an outlet in the tank wall for withdrawing the slurry formed by the slurry formation tool from the tank, wherein the outlet is in the form of a second flange through the tank wall. The two flanges on the tank wall can in one embodiment be a combined pipe in pipe flange. Searching outside patent literature has not revealed any prior art that appears to be of more relevance with respect to the present invention than U.S. Pat. No. 7,240,681 B2.

The objective of the invention is to provide a tool or equipment improving the removal of sand and other unwanted material from biogas reactors and other tanks and equipment subject to more or less the same problems as described above with respect to biogas reactors.

### SUMMARY OF INVENTION

The invention meets the objective by providing a tool for sand removal from biogas reactors and other containments, feasible for sand removal while the biogas reactor or other containment is in operation at elevated pressure and temperature.

The tool is distinctive in that it comprises:
a number of nozzles fluidly coupled to a high pressure liquid source for high pressure liquid injection, in operation directed at volumes with solid, semi solid or soft sand, wherein high-pressure liquid injection through the nozzles creates a flowable sand slurry,
a flowable slurry outlet opening facing the flowable slurry created by the nozzles, and
two flow paths, integrated in the tool, each flow path being fluidly coupled to one or both of the nozzles and the outlet opening, wherein a first flow path is for high pressure liquid to be injected through the nozzles and/or to create suction effect in the outlet opening, and wherein a second flow path is for removal of slurry collected through the outlet opening.

For many preferable embodiments, the tool further comprises an eductor, arranged in the second flow path with eductor suction inlet fluidly coupled to the outlet opening, or the eductor suction inlet is the outlet opening. Some preferable embodiments comprise several outlet openings. For an eductor, the motive fluid is a liquid and the produced vacuum or underpressure is used to draw in other liquid/fluid and sand and mix it with the motive liquid. However, the term ejector is often used synonymously, even though an ejector uses a gas as the motive fluid creating vacuum or underpressure.

The term high pressure liquid source means a source, such as the pressure side of a pump or a pressurized storage tank, enabling high pressure liquid injection through the nozzles, that is liquid injection at higher pressure than the pressure inside the reactor or containment from where sand shall be removed. This includes for example the high pressure feed flow 14 on FIG. 2, and the main feed flow 10 on FIG. 2, meaning that at least one such flow is obligatory.

For frequent sand removal, without very hard sediments, the main feed flow will typically be sufficient, with liquid overpressure typically up to about 2-10 bar above the reactor/containment pressure, and appropriate tools are designed accordingly, that is with only main feed flow or similar as preferable embodiments. For very hard sediments, a high pressure feed flow in addition or instead is required, with overpressure that can extend up to several hundred bar, and appropriate tools are designed accordingly, that is with one or several high pressure liquid conduits included in preferable embodiments.

Preferably, the tool comprises a single coupling part coupling the two flow conduits to a single tubing or pipe with two corresponding flow conduits, preferably by mating a pipe in pipe coupling part on the tool to a corresponding pipe in pipe coupling part in the end of a single pipe with two flow conduits.

Most preferably, the tool comprises only one coupling to a single pipe, such as a flexible pipe or flexible tube, which single pipe or tube includes at least two flow conduits. Such embodiments are preferable in general for operating in pressurized tanks, reactors, or equipment under elevated pressure and/or temperature since a single leak proof feedthrough can be arranged in an opening in a wall thereof.

Alternatively, the tool comprises several coupling parts, up to one coupling part for each conduit and/or function, such as two, three, four or more coupling parts.

Preferably, the tool comprises nozzles and outlet opening or eductor suction inlet in one and the same end of the tool and coupling part or parts in the opposite end of the tool. The outlet opening and/or the suction inlet to the eductor preferably is arranged coaxial to a longitudinal tool axis.

The tool preferably comprises nozzles directed in two or three directions, such as along or with direction components in x, y and z orthogonal directions, allowing positioning the tool in a tank within volumes of interest for removal of sand, wherein said positioning includes using the reaction forces created by operating the nozzles. In some preferable embodiments of the tool the nozzles 5 used for positioning may be fed by separate high pressure tubes or hoses.

Preferably, the tool in operation brakes up and fluidize sand settled in a tank or other equipment, disperses the sand in liquid, thinning the sand in the eductor and sucks out sand slurry.

One embodiment of the present invention is a combination of a tool of the invention coupled to a flexible tube or pipe brought sealingly through a tank wall opening or equipment wall opening, wherein the opening and related equipment comprises an injector head, packing box, lubricator, stuffing box, and/or slickline 15 seal and/or similar equipment through which the flexible tubing or a rigid pipe section is brought. Alternatively, the combination comprises two, three or more pipes or tubes brought sealingly through a tank wall. With the combination of the invention, while the tank or equipment is being emptied for sand, the tank or equipment, such as a biogas reactor, can be operated for biogas production as 20 normal, at normal operating parameters.

In the context of the tool, combination and use of the present invention, the term sand can mean not only sand but any process inactive material reducing the active volume in the tank or equipment, such as sand, stones, pebbles, egg 25 scales, shells, hard scales, concrete, ceramics, polymers, glass, metal, and also viscous foam or fluid that can be formed as unwanted by-products in tanks or equipment.

Preferable embodiments of the tool and/or combination of the invention May include one or more of the features as follows, in any combination:
one or more dilution nozzles, arranged to dilute the slurry;
one or more eductors/ejectors arranged to increase the rate of slurry formation in tanks or reactors with relative soft sand, that is loose sand or semi solid sand;
additional high pressure flow conduits, fluidly coupled to groups of nozzles or single nozzles, for allowing reaction forces to position the tool by operating the group of nozzles or nozzles, such as by operating a separate valve from outside the tank for each function;
nozzles arranged to overlap each other with respect to injection for dissolving and/or dispersing effect, in a circumferential volume and preferably in a spherical sector in front of, around of and partly rearwards from a front end of the tool;
the tool is a single unit;
the tool is in the form of a in substance cylindrical structure;
the tool is in the form of a in substance spherical structure;
the tool comprises a spherical front end coupled to or integrated with a cylindrical rear part;
similar or identical equipment for feed trough and sealing as used in the oil and gas industry for coiled tubing, coiled piping, wirelines and slicklines, are preferably used for the feed through in reactors or containers at elevated pressure and/or temperature;
buoyancy elements integrated in the tool for correct positioning when removing floating foam or other liquids/slurry floating on top in a reactor or container,
separate high pressure liquid feed pipes/conduits for each of nozzles to control x, y and or x movement of the tool by reaction forces from the injected high pressure liquid;
a bundle of separate tubes and/or pipe in pipe conduits arranged into a single rigid or flexible pipe section length through a single opening through a tank wall, wherein the bundles are moulded sealingly together inside the rigid or flexible pipe section, for example with a polymer, but can be arranged as one or several bundles or tubes outside the ends of the single rigid or flexible pipe section, wherein the rigid or flexible pipe section can be brought sealingly through a single seal through an opening in the reactor/containment/tank wall;
wherein the tool is brought into a tank or other equipment through an opening to a volume in the tank or other equipment where sand accumulate;
wherein the opening in the tank wall is at elevation above or below the sand level, wherein connected flexible tubing bends, allowing the tool to be directed in substance vertically in a tank, allowing x-y nozzle control and extending-retrieving tubing to control the positioning of the tool in the tank to cover all volumes with sand sediments.

### BRIEF DESCRIPTION OF DRAWINGS

FIG. 1 illustrates a tool and a combination according to the invention.

FIG. 2 illustrates flow directions in a tool of the invention.

FIG. 3 illustrates a tool of the invention.

### DETAILED DESCRIPTION OF THE INVENTION

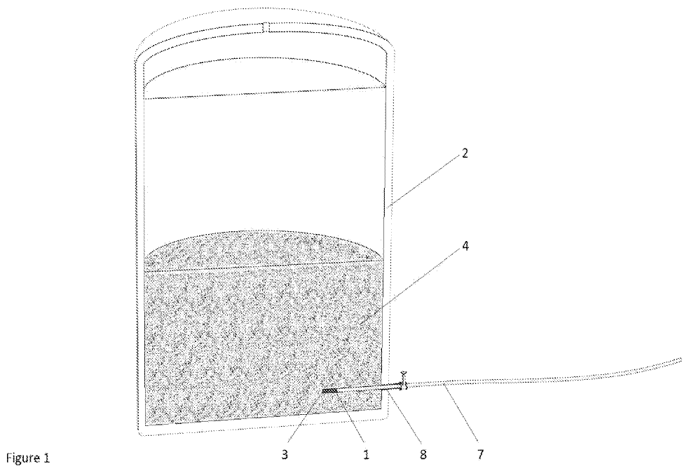

FIG. 1 illustrates a tool and a combination according to the invention.

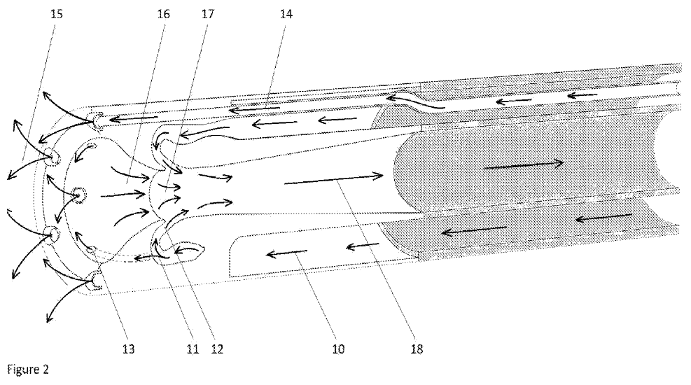

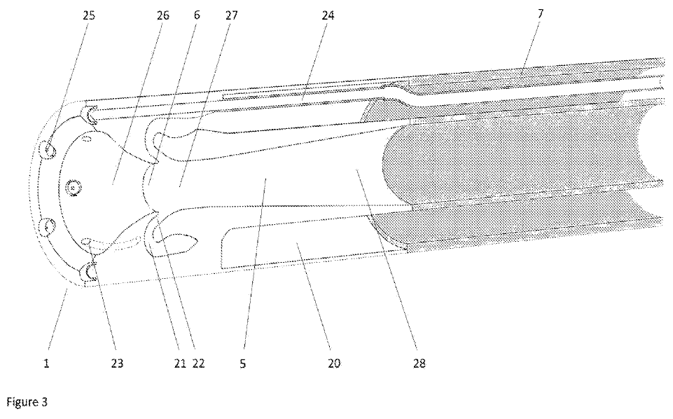

The tool 1 comprises a number of nozzles 3, in operation directed at volumes 4 with solid, semi solid or soft sand, in the lower part of a biogas reactor 2. The effect of operating the nozzles by high-pressure liquid injection is to create a flowable sand slurry. The sand slurry is brought out through the second flow path, via the eductor of the tool, wherein the eductor is arranged with at least one suction opening facing the flowable slurry created by the nozzles. The tool comprises two flow paths, integrated in the tool, each flow path being fluidly coupled to one or both of the nozzles and the eductor, wherein a first flow path is for high pressure fluid to be injected through the nozzles and/or to create suction effect in the eductor, and wherein a second flow path is for returning slurry collected by operating the eductor. The detailed design of the tool embodiment illustrated, is easier understood by referring to FIGS. 2 and 3.

FIG. 2 illustrates flow directions in a tool of the invention. More specifically, the arrows illustrate flows as follows:
10. Main Feed Flow
11. Splitting Flow/Dividing Flow
12. Eductor Feed Flow/Eductor Inlet Flow
13. Dilutor Feed Flow/Dilution Feed Flow
14. High Pressure Feed Flow/Injection Feed Flow
15. Nozzle Flow/Dissolver Flow/Dissolving Flow
16. Suction Flow/Eductor Suction Flow
17. Eductor Mixing Flow/Eductor Mixing and Diluting Flow
18. Eductor Outlet Flow.

FIG. 3 illustrates the tool embodiment of the invention for which flows are illustrated in FIG. 2. More specifically, the tool comprises:
20. Main Feed Conduit
21. Splitting Flow Zone/Dividing Flow Zone/Flow Split Zone
22. Eductor Feed Conduit/Eductor Inlet
23. Dilutor Feed Conduit/Dilution Feed Conduit
24. High Pressure Feed Conduit/Injection Feed Conduit
25. Nozzle/Dissolver
26. Suction Zone/Eductor Suction Zone
27. Eductor Mixing Zone/Eductor Mixing and Diluting Zone
28. Eductor Outlet

FIG. 1 illustrates a typical biogas reactor in cross section and a typical tool of the invention for removing sand from the biogas reactor, in longitudinal section. A typical tool embodiment is illustrated in detail in FIGS. 2 and 3, in longitudinal section. For other uses of the tool than for removing sand from a biogas reactor in operation, the slidable leak proof penetration of a tank wall may not be necessary. For tanks or containments not under elevated pressure and/or temperature, or not in operation at all, the tool can be lowered down to the sand filled volumes to be emptied by a hoist or winch system. By controlling the outlet opening flow and tool positioning, with or without an eductor integrated in the tool, unwanted fluid can be sucked into the tool and be removed from the reactor or other containment. Numerous further embodiments of the tool and the combination of the invention are possible, and uses, for example by combining features as described or illustrated herein in any operative combination as foreseeable by a person of ordinary skill in the art, and all such embodiments are embodiments of the present invention.

1-10. (canceled)

11. A tool for sand removal from biogas reactors and other containments, feasible for sand removal while the biogas reactor or other containment is in operation at elevated pressure and temperature, the tool comprising:
a number of nozzles fluidly coupled to a high-pressure liquid source for high pressure liquid injection, in operation directed at volumes with solid, semi solid or soft sand in front of the tool, wherein the nozzles are arranged to overlap each other with respect to injection effect, in a circumferential volume, and wherein high-pressure liquid injection through the nozzles creates a flowable sand slurry;
a flowable slurry outlet opening facing the flowable slurry created by the nozzles;
one or more dilution nozzles, arranged to dilute the slurry; and
two flow paths, integrated in the tool, wherein a first flow path is for high-pressure liquid to be injected through the nozzles and/or to create suction effect in the outlet opening, and wherein a second flow path is for removal of slurry collected through the outlet opening.

12. The tool according to claim 11, comprising nozzles and outlet opening or ejector suction inlet in one and the same end of the tool and coupling part or parts in the opposite end of the tool.

13. The tool according to claim 11, comprising an ejector, arranged in the second flow path with ejector suction inlet fluidly coupled to the outlet opening.

14. The tool according to claim 11, wherein the tool is coupled to a flexible tubing comprising at least two flow conduits.

15. The tool according to claim 11, comprising nozzles directed in two or three directions, allowing positioning the tool in a tank within volumes of interest for removal of sand, wherein said positioning includes using the reaction forces created by flow through the nozzles.

16. The tool according to claim 11, as coupled to a flexible tube or a rigid pipe section brought sealingly through a tank wall opening or equipment wall opening, wherein the opening and related equipment comprises an injector head, packing box, lubricator, stuffing box, and/or slickline seal and/or similar equipment through which the flexible tube or the rigid pipe section is operatively arranged.

17. The tool according to claim 16, wherein the tool position is controlled by operating the injector head for running in or retrieving tube or pipe in the tank or equipment, and operating nozzles in selected directions, while the slurry formation and removal of the formed slurry is controlled by operating or controlling the fluid flow through the nozzles and the outlet opening.

18. Use of the tool according to claim 11, for injection of high-pressure fluid for slurry formation into a tank or other equipment and/or removal of material from a tank or other equipment.

19. Use of the tool according to claim 18, for slurry formation of sand and other material not active in biogas formation in a biogas tank, and removal of the slurry created, while the biogas tank is in operation for biogas production.

20. Use of the tool according to claim 18, for removal of a top layer formed by flotation or chemical processes in a tank or other equipment, by sucking in said material through the outlet opening, with or without an ejector.
